(19) United States
(12) Patent Application Publication
(10) Pub. No.: US 2008/0243102 A1
(43) Pub. Date: Oct. 2, 2008

(54) Apparatus for segmental varicose sclerosis

(76) Inventor: Banning Gray Lary, Miami, FL (US)

Correspondence Address: MCHALE & SLAVIN, P.A., 2855 PGA BLVD, PALM BEACH GARDENS, FL, 33410, US

(21) Appl. No.: 12/056,005

(22) Filed: Mar. 26, 2008

Related U.S. Application Data
(60) Provisional application No. 60/920,291, filed on Mar. 27, 2007.

(51) Int. Cl. A61M 31/00; A61M 25/09

(52) U.S. Cl. 604/509; 604/164.09

(57) ABSTRACT

The present invention relates to a minimally invasive apparatus and method of use to correct varicose veins by sclerosis. The apparatus comprising a semi-flexible first catheter having a proximal end, a distal end, and an outer surface. The first catheter also has a lumen area extending the length of the catheter and capable of receiving a guide wire. The proximal end is fitted to receive an inflation device and/or a closure device. At the distal end, a collapsible occlusive balloon is attached. Additionally, the apparatus has a second catheter having a proximal end, a distal end, an outer surface. The second catheter also has a lumen area extending the length of the second catheter. The second catheter has a larger lumen than the first catheter, allowing the first and second catheters coaxial alignment. The distal end of the second catheter adapted to from a sealing engagement with the periphery of the first catheter, allowing the second catheter to movably extend over and retract from the first catheter. Attached to the second catheter is a reservoir for dispensing a treatment agent into the lumen. The second catheter is adapted to connect to the reservoir. The treatment agent is dispensed to the vein via one or more side openings.

### CROSS REFERENCE TO RELATED APPLICATION

This application claims priority under 35 U.S.C. § 119(e) to U.S. Provisional Application No. 60/920,291, filed on Mar. 27, 2007, the contents of which are herein incorporated by reference.

### FIELD OF THE INVENTION

The invention relates to the treatment of venous insufficiency; more particularly, this invention relates to a minimally invasive procedure and apparatus to correct varicose veins by sclerosis.

### BACKGROUND OF THE INVENTION

Varicose veins are a common, generally non-life threatening medical condition associated with enlarged veins. Although they may be found on any part of the body, they are most often associated with the veins located along the leg from the groin area to the ankle. Varicose veins are mostly associated with the veins of the superficial venous system, particularly the great saphenous vein, and the smaller tributary branches that feed into them. Visual appearance on the leg can range from small spider veins to large subcutaneous grapelike clusters. Varicose veins often are blue or dark purple in appearance, have a twisted, rope-like appearance and can be raised against the skin surface. In most instances, varicose veins generally do not cause serious medical problems and are more of a cosmetic nuisance. However, some varicose vein sufferers develop symptoms such as deep pain or aching feeling, heaviness, burning or tingling sensations, swelling and tenderness around the vein and may signal the beginning of more complicated medical issues. In more severe cases, varicose veins can be associated with skin ulcers inflammation and blood clots, requiring immediate attention.

Varicose veins are a relatively common problem affecting all individuals. Several risk factors tend to increase the chances of developing the condition. Resulting from numerous years of the wear and tear associated with blood flow and pressure placed on the body, varicose veins tend to be found in older individuals. A person's gender may also increase ones risk, with women suffering from the condition in greater numbers then men. Other risk factors include obesity, genetics, and extended periods of standing. Increasing blood flow through exercise, watching one's weight and avoiding prolonged standing may help prevent the disease.

The circulatory system is made up of vessels and muscles which help regulate blood flow through the entire human body. It is responsible for moving blood to and from the numerous tissues within the body. The circulatory system is characterized by arteries which take oxygen rich blood from the heart to body tissues. Veins return waste-rich blood from the body to the heart which is eventually re-circulated. Within the body there exists many branches of arteries and veins specific to certain areas. Of importance to varicose veins are those branches of the circulation system associated with the lower limbs.

Veins of the lower limbs consist of the superficial veins and deep veins. The superficial veins run through the body close to the surface; deep veins traveling through the body at a distance from the surface are often associated with arteries and carry most of the blood. Connecting the superficial and deep veins are the perforating veins. In the lower limb system, the perforating veins pass through the deep fascia at mid-thigh, knee and ankle.

The principal veins running superficially up the leg are the saphenous veins. The great saphenous vein, also called the large saphenous vein, runs from the foot and passes through a fibrous membrane, called the saphenous opening, located in the broad fascia of the thigh. The vein eventually joins the femoral vein at the femoral triangle. Along the length of the vein, it receives several small branches, or tributaries. The small saphenous veins, also known as the lesser saphenous vein, is a relatively large vein that runs behind the outer malleolus, travels up the back of the leg and drains into the popliteal vein in the popliteal space, the space behind the knee.

One part of the deep vein system of the lower limbs is the femoral vein. The femoral vein, the largest vein in the groin, is closely associated with the femoral artery. It begins in the adductor canal and ends at the inferior margin of the inguinal ligament. The vein is connected to the common iliac veins through the external iliac vein. Several large veins drain into the femoral, including the popliteal vein, profunda femoris vein, and the great saphenous vein.

As a result of being located farthest from the body, veins, which function to return blood back to the heart, associated with this system must pump blood long distances against the forces of gravity. To help accomplish this, movement of the diaphragm creates a negative pressure helping the blood travel back to the heart from the legs. In addition, muscle contractions act as pumps pushing the blood through the elastic vein walls. Associated with the blood flow through the arteries are small, one-way valves. These one-way valves allow upward flow of blood by opening as the blood travels in the direction toward the heart. The valves close to prevent blood from flowing in the backwards direction. Given the long distances and constant stress the lower extremities are under, the veins are under constant strain. When the valves are strong and functioning properly, back-flowing blood is not a problem. As individuals age, the valves begin to malfunction. Veins begin to loose their elastic nature, causing stretching of the vein. This stretching results in weakening valves, allowing blood which normally travels in the upward direction, to flow in the backwards direction. When blood flows backwards, it begins to pool in the vein thereby causing a larger and varicose vein.

Whether for cosmetic or medically-necessary reasons, individuals have several options for treating varicose veins. Vein stripping has historically been the standard treatment for varicose veins, particularly for use on larger veins. This process requires removing the vein through small incision sites. Removal of the vein does not effect the overall blood circulation as other veins in the system quickly adapt. Typically performed in a hospital setting under general anesthesia, physicians begin the process with an incision near the groin to expose and ligate the great saphenous vein and connecting, or tributary, veins. A surgical instrument called a stripper is inserted into the vein along the length of the thigh to the knee where the instrument is removed through a second incision in the skin. The saphenous vein is tied to the stripping instrument. The vein is removed from the body by pulling on the stripper. As the surgeon pulls down the instrument traveling the length of the vein, the vein turns inside out and is removed at the second incision site. Tributary veins may be removed by breaking off as the vein is stripped from the body or by way of tiny incisions, a process known as ambulatory phlebectomy.

Although this procedure can effectively be used to remove larger varicose veins, stripping has several disadvantages. Stripping is a surgical procedure and complications associated with anesthesia are possible. In addition to the pain associated with the groin surgery, as with any surgery, development of internal bleeding, infection, blood clots, and nerve damage are possible risks. Post operative pain, discomfort, tenderness and extended recovery time limits patient activities post surgery while recovering. Some patients require several days to recuperate, while other patients take several weeks before returning to normal activities. Patients may suffer from bruising and discoloration. Finally, recurrence of varicose vein and scarring are possible long term problems associated with the procedure.

An alternative to vein stripping and the current preferred method of treatment is sclerotherapy. This process is most successfully used to treat smaller varicose veins. Physicians inject a significant amount of a sclerosing agent, a chemical, into the vein wall causing irritation and damage to the wall at the injection site. The sclerosing agent eventually causes collapse of the vein as the body eventually absorbs the vein. Other veins in the body absorb the blood flow which would have returned to the heart via the treated vein. However, the sclerosing agent is not effective further down the injection site as the chemical gets diluted within the large volume of blood. Although this process is a preferred treatment, there are several disadvantages. In performing the surgery, the surgeon must evenly dispense the agent to the vein wall without using toxic levels. Only a small area can be injected; large areas can not be effectively utilized because of the possible dilution caused by blood flow in the vein. Recurrence of the varicose veins often occurs in patients, resulting in additional injections. If the sclerosing agent concentration used in the surgery is too weak, incomplete action results and recannulization may occur. If the agent is too concentrated, the vein of interest will be destroyed but the possibility on injury to the non-varicose veins in the surrounding area increases. In addition, improper injection could result in the uneven destruction of the vein. Moreover, complications such as scarring and necrosis of tissue can result if the physician performing the injections does not get the sclerosing agent into the vein.

Therefore, what is needed is a minimally invasive apparatus and method of use which allows the surgeon to utilize minimal injection sites into the skin while allowing the surgeon to deliver sclerosing agents to the vein at multiple sites without the risk of diluting the sclerosing agent concentration.

More recently, additional minimally invasive techniques have been developed. Catheter assisted procedures, known as radiofrequency ablation have become a possible alternative to stripping and sclerosis. In this procedure, a physician inserts a catheter into the vein. The catheter contains electrodes which are used to pass RF energy through the vein tissue. The radiofrequency therapy causes vein wall damage through heat and scarring, eventually causing the vein to collapse and seal. As compared to sclerotherapy, this procedure can be more cumbersome, slower and more painful. Another disadvantage of radiofrequency ablation is that the vein is destroyed only at the vein wall-electrode contact point, increasing the chances destroying other tissues and recannulization. Possible side effects to the procedure include skin burns, possible nerve damage and formation of blood clots.

In addition to radiofrequency ablation, physicians may use laser assisted procedures. During this procedure, laser energy is emitted from a thin laser fiber inserted into the vein. Short pulses of laser energy are delivered, damaging the vein which eventually shrinks the vein until blood can no longer pass through. The veins slowly fade and eventually disappear. Careful attention with regards to the duration and intensity of the treatment must be monitored, as accidental laser contact outside the body is a possibility. Side effects for this procedure include bruising, skin burns, nerve damage and formation of blood clots. Moreover, the high cost associated with the procedure may be prohibitive.

As used herein, percutaneous refers to any medical procedure that provides access to blood vessels, inner organs or various tissues and is performed using a needle puncture to the skin. This technique is minimally invasive as compared to open surgery in which surgeon exposes the inner body organs or tissues typically with the use of a scalpel. This type of procedure is often used in vascular procedures and begins with a needle catheter entering a blood vessel. A guide wire may then be inserted into the lumen of the needle allowing other surgical instruments, such as, but not limited to, catheters or endoscope instruments, to be placed into the vessel. Percutaneous procedures are advantageous because they are less invasive and are associated with less risk of infection and pain associated with open incision sites.

As used herein, a Kirschner wire, also called a K-wire, is a thin, rigid wire. They are typically used in orthopedic surgeries to stabilize bone fragments. K-wires can be placed through the skin and are drilled through bone, holding bone fragments in place.

### DESCRIPTION OF THE PRIOR ART

U.S. Pat. No. 5,370,617 discloses a dilation catheter capable of relieving an obstructed artery while allowing blood to flow past the obstruction. While the patent discloses administering treatments while maintaining blood flow. When placed in the vein and extended, the balloon is the treatment. Openings in the catheter allow for blood to continually flow past the obstruction

The use of a catheter system and methods of use for delivery of therapeutic agents is not new to the art. U.S. Pat. No. 6,264,633 discloses a balloon catheter for transventricular blocking of an aorta lumen. Then apparatus has a inner shaft used for a guide wire, a first lumen used for dispensing, and a second lumen used for suctioning blood. The balloon is inserted into the aorta, and a cardioplegia solution is discharged through an outlet of a lumen while blood is suctioned into a heart-lung machine by way of a second outlet from a second lumen.

U.S. Pat. No. 5,840,066 discloses an infusion catheter having an inflatable balloon, a blood passage having two openings for blood to pass through the balloon, and two lumens. One lumen is used to expand the balloon; the second lumen used to dispense medicine to a designated part of a blood vessel. In dispensing the medicine, the entire catheter moves. Similarly, U.S. Pat. No. 6,544,273 discloses a drug delivery device. The device has an inflatable balloon attached to the distal end of an elongated shaft. A second tube houses a guide wire. The balloon has a plurality of openings which provide for dispensing of a treating agent. Although the '223 patent discloses use of two tubes, the tubes are in a fixed position relative to each other.

U.S. Pat. No. 7,077,836 to Lary discloses an apparatus for delivering an intravascular drug to a vein. The apparatus has three concentric tubes. The innermost tube has an inflatable balloon attached at the distal end of an elongated shaft. The intermediate tube has a self-expanding balloon. Expansion of the balloon occurs by movement of the outer most tube relative to the intermediate to the intermediate tube. Treating agents are dispensed through pores located on the innermost tube or on the balloon. The veins may be sclerosed as the self-expanding balloon is pulled along the vein.

Improvements to the apparatus of the '836 patent resulted in subsequent patent applications. U.S. Patent Application Publication 0149218, and the corresponding PCT Application PCT/US04/03249, disclose a method and apparatus for treating the interior of a blood vessel. The apparatus has two catheters, the second catheter movable within the first catheter. The second catheter further having at least one perforation adapted to align sequentially with the plurality of perforations located on the first catheter. In addition, several related applications disclose improvements to or various methods and apparatus for dispensing sclerosing agents within an occluded blood vessel, such as U.S. Patent Application 2005/0113798, US Patent Application 2005/0107738, U.S. Patent Application 2006/0189929, U.S. Patent Application 2006/0189930, U.S. Patent Application Publication 2003/0120256 and PCT/US2002/021132.

However, it is desired to have additional apparatus and methods of treating varicose veins, and in particular in a minimally invasive manner which segmentally delivers sclerosis agent to the vein wall while essentially completely preventing blood flow and dilution problems.

### SUMMARY OF THE INVENTION

The present invention relates to a minimally invasive apparatus to correct varicose veins by sclerosis. The apparatus comprising a flexible first catheter having a proximal end, a distal end, and an outer surface. The first catheter also has at least one lumen area extending the length of the catheter and capable of receiving a guide wire. Furthermore, it is positioned along the longitudinal axis of the guide wire. At or near the proximal end the first catheter is fitted to receive an inflation device and/or a closure device. At the distal portion, a collapsible occlusive balloon is attached. Alternatively, the first catheter may also have two or more lumens. The first lumen may be adapted to receive the guide wire while the second lumen could be adapted to receive a balloon inflating substance dispensed from the inflation device. In yet another alternative embodiment, a first and second catheter may be provided as a vein sclerosing catheter assembly, pre-assembled prior to insertion into the vein.

As indicated above, the apparatus has a second catheter having a proximal end, a distal end, and an outer surface. The second catheter also has a lumen area extending the length of the second catheter. The second catheter has a larger lumen than the first catheter, allowing the first and second catheters coaxial alignment. It is positioned longitudinally and in juxtaposed relationship along the longitudinal axis of the first catheter. The distal end of the second catheter contains an opening, allowing it to movably extend over and retract from the first catheter but is adapted for reciprocating longitudinal movement about the first catheter while maintaining the sealing engagement with the periphery of the first catheter. Attached to the second catheter is a reservoir for dispensing a treatment agent into the lumen. The reservoir is fluidly coupled to the proximate end of the second catheter. The treatment agent is dispensed to the vein via one or more side openings which may be radially and circumferentially positioned about the distal end.

The present invention also relates to a minimally invasive method to correct varicose veins by sclerosis using the apparatus as described above. According to one embodiment of the method, a patient is prepared for surgery. While in the supine position, anesthesia is administered and the surgeon forms a means of ingress into the skin and vein. A guide wire is inserted into the vein and positioned at a desired location. A first catheter having an occlusion balloon attached to the distal end is passed over the guide wire and placed in the desired position. Using a liquid or gas substance, the balloon is expanded. A reference mark is placed on the first catheter at the distal entrance site to the vein. The first catheter is occluded to maintain the inflation of the balloon. A second catheter is placed over the first catheter, resting at the distal part of the occlusive balloon. In an alternative embodiment, the surgeon may provide a vein sclerosing catheter assembly in which the first catheter and second catheter is pre-assembled prior to insertion. The patient's leg is elevated and placed on a bolster. A sclerosing agent is injected into the lumen of the second catheter from a reservoir connected to its proximal end and dispensed to the vein through openings of the second catheter. The surgeon then massages the area. After injecting the sclerosing agent, the second catheter is partially retracted and external means of compression, i.e. elastic wrappings are applied. Injection, massage, retraction and external means of compression, are repeated until the vein is completely treated. The second catheter is removed from the body. The surgeon then collapses the inflated balloon, first removes the first catheter and then removes the guide wire. After removal of the apparatus, the incision site is closed. Post-operative treatment is then applied.

In a second embodiment of the treatment, the guide wire is passed proximal within the great saphenous vein and placed at the junction of the saphenous and femoral veins. The first catheter is then placed over the guide wire to the saphenous-femoral junction. The occlusion balloon is inflated, occluding the opening between the saphenous and femoral veins. A second catheter is placed over the first catheter, resting at the distal part of the occlusive balloon. Alternatively, the surgeon may provide a vein sclerosing catheter assembly in which the first catheter and second catheter is pre-assembled prior to insertion. The patient's leg is elevated and placed on a bolster. A sclerosing agent is injected into the lumen of the second catheter from a reservoir connected to the proximal end and dispensed to the vein through openings of the second catheter. The surgeon massages the area. After injecting the sclerosing agent, the second catheter is partially retracted and external compressions are applied. Injection, massage, retraction and external compressions are repeated until the vein is completely treated. The second catheter is removed from the body. The surgeon then collapses the inflated balloon, first removes the first catheter and then removes the guide wire. After removal of the apparatus, the incision site is closed. Post-operative treatment is then applied.

Accordingly, it is a primary objective of the instant invention to provide a minimally invasive apparatus to correct varicose veins by sclerosis.

It is an additional objective of the instant invention to provide a minimally invasive pre-assembled apparatus to correct varicose veins by sclerosis.

It is a further objective of the instant invention to provide a minimally invasive procedure to correct varicose veins by sclerosis.

It is still further an objective of the instant invention to provide minimally invasive procedure and apparatus to correct varicose veins by sclerosis without using extremely toxic concentrations of sclerosing agents.

It is yet another objective of the instant invention to provide a minimally invasive procedure and apparatus to correct varicose veins by sclerosis wherein the sclerosing agent is does not get diluted during the treatment procedure.

It is a still further objective of the invention to provide a minimally invasive procedure and apparatus to correct varicose veins by segmentally applying a sclerosing agent to a particular vein.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 illustrates a patient's leg depicting varicose veins from the groin to the ankle.

FIG. 2 is an expanded view of the varicose veins, showing the characteristic irregular shape.

FIG. 3 illustrates several components of the limbic circulatory system, including the saphenous vein, femoral vein and saphenous-femoral junction.

FIG. 4A is an illustration of the sclerosing apparatus of the instant invention.

FIG. 4B is a cross section of the segmentally sclerosing apparatus, illustrating the coaxial alignment of the first and second catheters.

FIG. 4C illustrates an additional embodiment of the segmentally sclerosing apparatus whereby the first catheter has two or more lumens.

FIG. 5A is an illustration of the initial incision site located at the distal portion of the leg.

FIG. 5B illustrates insertion of the first catheter at the incision site and resting at the saphenous-femoral junction.

FIG. 5C illustrates insertion of the second catheter at the saphenous-femoral junction.

FIG. 6A illustrates the fully assembled apparatus placed at the initial injection site.

FIG. 6B illustrates the second catheter partially retracted from the first injection site and positioned at the second injection site.

FIG. 6 C illustrates the second catheter partially retracted from the second site and positioned at the third injection site.

### DETAILED DESCRIPTION OF THE INVENTION

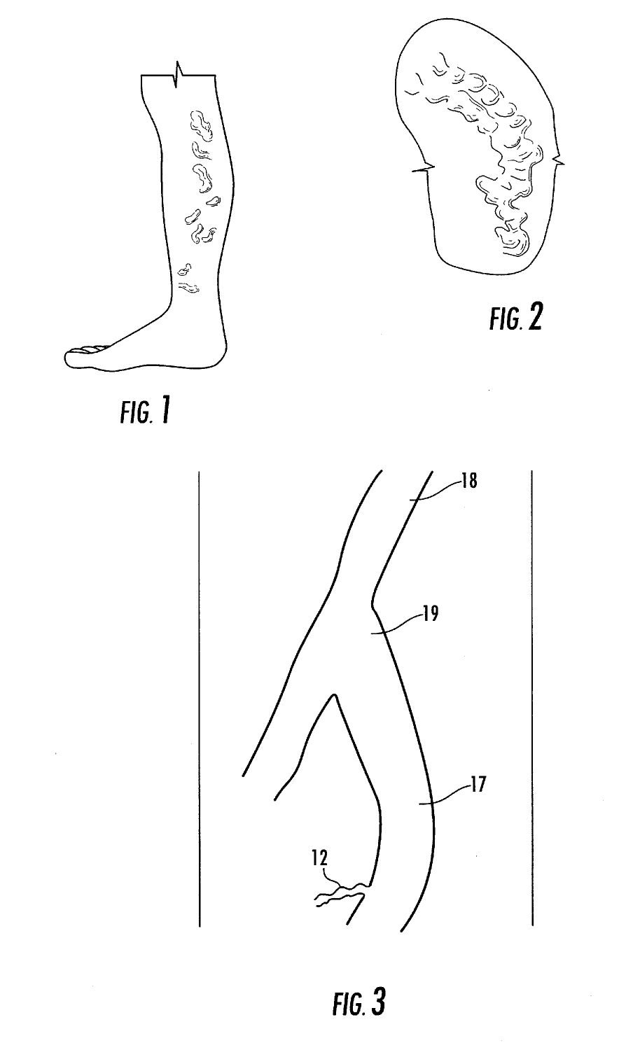

As described earlier, varicose veins are most commonly located on the leg. FIG. 1 shows an illustration of varicose veins located along the length of the leg from the groin area to the ankle. FIG. 2 is an expanded view of the veins and illustrates the characteristic twisted, rope-like appearance of the veins. Because the veins are associated with the superficial venous system, treating varicose veins involves the removal of or insertion of instruments into the vein. In particular, FIG. 3 illustrates the two vein systems of the lower limb system, the superficial vein, such as the great saphenous vein (17) and the deep vein, for example, the femoral vein (18). The great saphenous vein (17) deposits into the femoral vein (18) at the saphenous-femoral junction (19). In addition, several smaller veins, known as tributaries (12) deposit into the great saphenous vein (17).

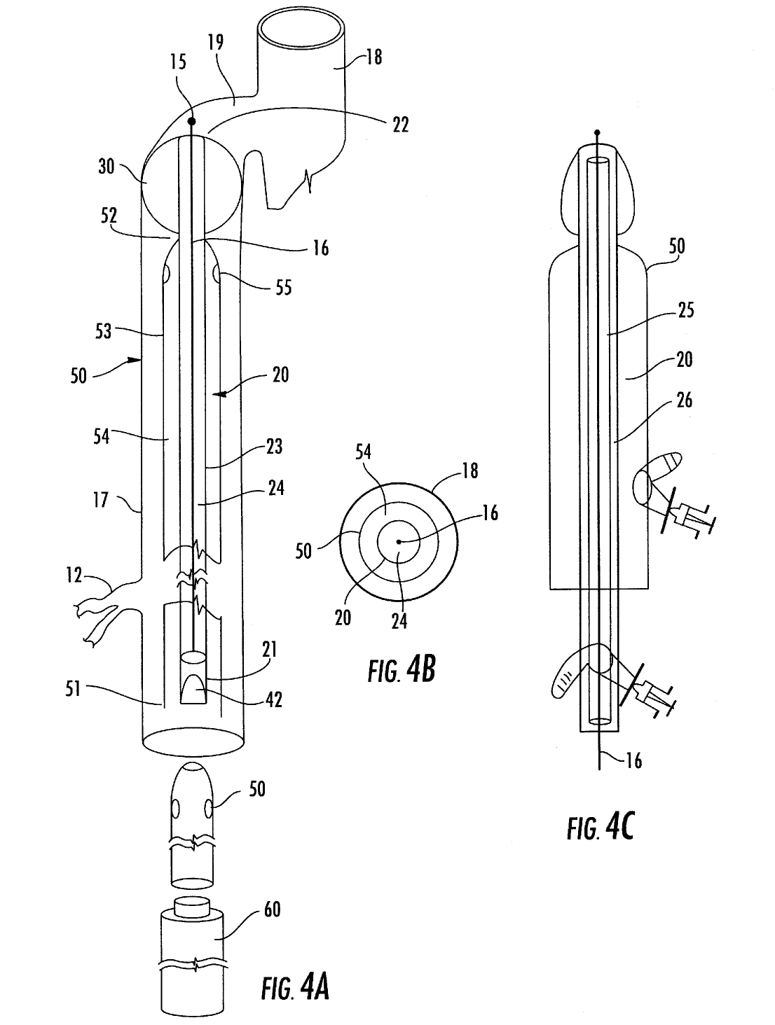

Referring to FIG. 4A, the apparatus for the segmental varicose sclerosis has a guide wire 16 and guide wire having a tip 15. The apparatus further has a flexible first catheter 20. The first catheter 20 has a proximal end 21, a distal end 22, an outer surface 23, and a lumen area 24. The lumen area 24 extends the length of the first catheter 20 and is capable of receiving a guide wire 16 and guide wire tip 15. The first catheter is positioned along the length of the longitudinal axis of the guide wire 16. Near the proximal position, catheter 20 is fitted to receive an inflation device 40 and/or a closure device 42. Although FIG. 4 exemplifies the inflation device 40 as a syringe, it is appreciated that inflation device 40 can be any device that can dispense a liquid or gas substance, such as but not limited to, a pump, bulb, valve, inlet port or generator.

Attached to and located at the distal end 22 of the catheter 20 is a collapsible occlusive balloon 30. Although FIG. 4A illustrates the balloon 30 in the deflated position, one skilled in the art would recognize that the collapsible occlusive balloon could also assume an inflated, extended position. The collapsible occlusive balloon 30 is fitted to receive any type of inflation substance fluidly communicated through the lumen area 24 of the first catheter 20.

The apparatus further has a second catheter 50. The second catheter 50 has a proximal end 51, a distal end 52, an outer surface 53, and a lumen area 54. The lumen area 54 is larger than the first catheter 20 and extends the length of the second catheter 50. The distal end 52 has an opening to allow the second catheter 50 to longitudinally extend over the longitudinal axis of the first catheter 20. In this alignment, the first catheter 20 and second catheter 50 are coaxially aligned and in juxtaposed relationship with the second catheter capable of moving over and retracting from the first catheter 20. FIG. 4B is a cross-section view of the apparatus and illustrates the coaxial alignment and positioning of the guide wire 16, first catheter 20, second catheter 50 and vein 11. Despite the second catheter receiving and extending over the first catheter, the second catheter is further adapted for reciprocating longitudinal movement about the first catheter 20 while maintaining the sealing engagement. Such sealing engagement is illustrated by, albeit not limited to, an impermeable seal created through a tight fit or use of specific materials which prevent movement of fluids in the inward or outward direction. Located at or near the proximate end 51 of the second catheter 50 is a reservoir device 60 which is fluidly coupled to the proximate end of the second catheter. The reservoir device is capable of holding and dispensing a treatment agent into the lumen 54 of the second catheter. The second catheter further has one or more openings 55. The lumen 54 is fluidly coupled to the radially and circumferentially spaced openings 55 at the distal end 52 of the second catheter 50 and dispenses fluids outwardly to the targeted area, such as a vein. Although FIG. 4 exemplifies the reservoir 60 as a syringe, it is appreciated that reservoir 60 can be any device that can dispense a liquid, such as a pump, bulb, valve, inlet port or generator.

In an alternative embodiment as seen in FIG. 4C, the apparatus for segmental varicose sclerosis provides that the first catheter 20 may have two or more lumens. The first lumen 25 is adapted to receive the guide wire 16. The second lumen is 26 adapted to receive a balloon inflating substance dispensed from the inflation device. The first catheter also contains an inflation device and a closure device. Although not limited to the illustration, the inflation device can be a syringe inserted into an inlet port. Moreover, the second lumen 26 corresponding to the first catheter 20 can be sealed with a stopcock. The second catheter also has a reservoir for dispensing a treatment agent and can be sealed on the first catheter.

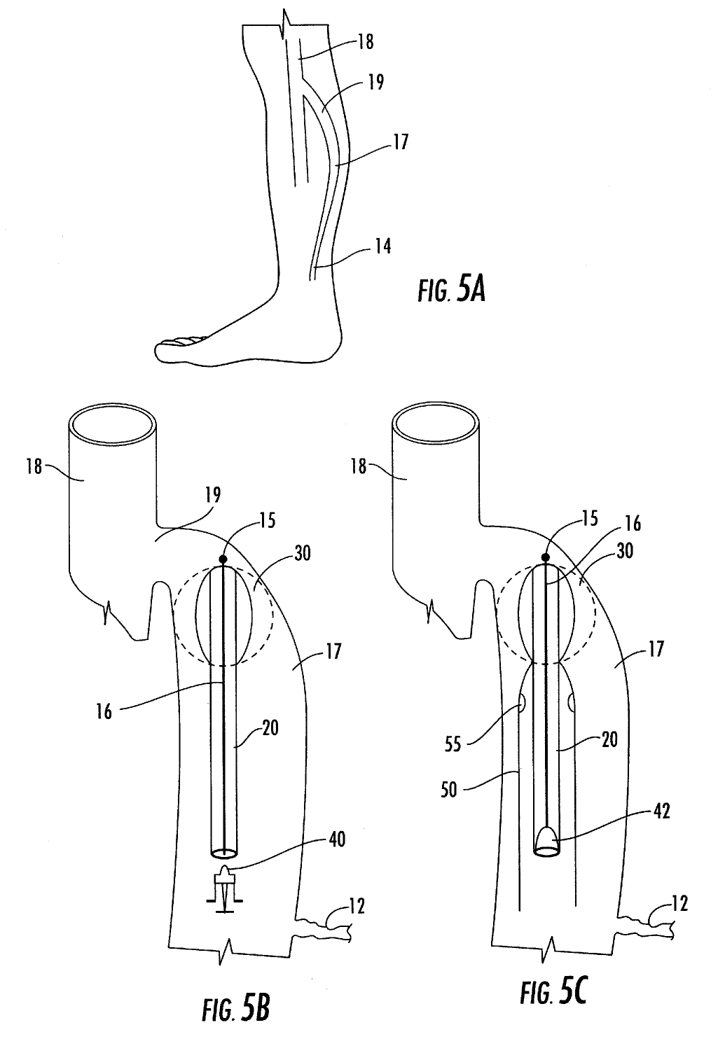

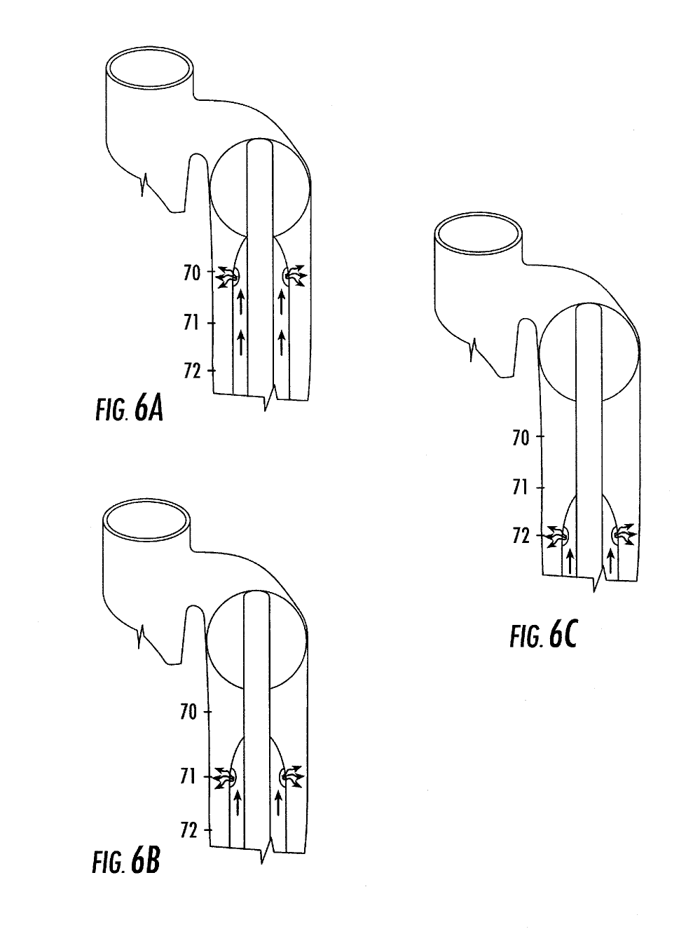

An exemplary method of treating varicose veins using the embodiments of the apparatus as previously described is illustrated in FIGS. 5-6. With the patient in the upright position, a marking system is applied to the skin superimposing the areas corresponding to the varicose veins. The patient is placed in the supine position and face up. A small amount of topical anesthesia is administered to the area over the distal portion of the varicose vein. The incision and opening of the skin is located at the distal portion of the leg. In one embodiment of the invention, the incision may be made at any vein in the body. In a second embodiment, as illustrated in FIG. 5A number 14, the incision is made in the distal saphenous vein wall. Additionally, the surgeon may form any means of ingress into the skin and veins, such as insertion of the apparatus precutaneously. A guide wire 16 is passed proximal within the saphenous vein and positioned at the junction 19 of the saphenous vein 17 and femoral vein 18. The first catheter 20 as described above is passed along the longitudinal axis of the guide wire 16. The occlusive collapsible balloon 30 is placed at or near the saphenous-femoral junction 19. It should be understood that placement of the catheter at the saphenous-femoral junction is not a limiting embodiment. One of ordinary skill can envision placement of the balloon anywhere within the vein or body cavity.

When the balloon is placed in the desired position, it is expanded using the inflation device 40 via dispensing a liquid or gaseous substance through the lumen 24 of the first catheter 20. As visualized in FIG. 5C, expansion of the balloon occludes the opening between the proximal saphenous vein 17 and the femoral vein 18, preventing blood flow from the saphenous vein into the femoral vein. To determine the proper positioning and inflation, the surgeon can either palpate the inflated balloon or verify through ultrasound or x-ray. At the distal entry site into the vein a reference mark is placed on the first catheter 20 for subsequent reference. The closure device, see FIG. 5C, 42, maintains the inflation of the balloon by sealing some or the entire first catheter 20. Although FIG. 5C illustrates an indwelling plug, one skilled in the art can appreciate that the closure device could be any such device that prevents escape of the liquid or gas, such as, but not limiting to, a ligature, plastic adhesive or stopcock. Upon sealing the closure device, the second catheter 50 is inserted into the vein, longitudinally extending along the longitudinal axis of the first catheter 20 and resting at the distal end 22 below the collapsible occlusive balloon 30, coaxially aligning with the first catheter 20. The distal end 52 of the second catheter 50 is adapted for longitudinal movement about the first catheter while maintaining the sealing engagement with the periphery of the first catheter 20, preventing fluid from exiting the catheter at the distal tip. In an alternative embodiment, the first and second catheter maybe provided as a vein scleroing catheter assembly which is pre-assembled prior to percutaneous placement.

Once the second catheter is in place, the leg of the patient is elevated thirty to forty degrees and placed in a bolster. Specified amounts of sclersosing agents are dispensed from the reservoir 60. Dispensed sclerosing agent is received by the lumen 54 of the second catheter 50 which is fluidly coupled to radially and circumferentially spaced openings 55 at the distal end of the second catheter 50. Sclerosing agents dispersed through the openings are distributed to the saphenous vein 18 wall. Although one skilled in the art would recognize the appropriate sclerosing agent, detergents such as sodium morrhuate, ethanolamine oleate, sodium tetradecyl sulfate, polidocanol, hypertonic solutions such as hypertonic saline, sclerodex (a mixture of 25% dextrose and 10% sodium chloride and small amount of phenethyl alcohol), polyiodinated iodine (mixture of elemental iodine with sodium iodide with small amount of benzyl alcohol) or chemical irritants, such as glycerin, urea, quinine, and sodium salicylate. Other sclerosing agents may include alcohol, bleomycin, picibanil and ethibloc.

After injection of the measured amount of sclerosing agent, the area is messaged by the surgeon. The second catheter 50 is then partially retracted to the next area for dispersing of the sclerosing agent, as represented in FIG. 6, 70-72. The first catheter 20 remains in place. Immediately after the injection, external compressions or dressings are applied to the corresponding area. As illustrated in FIGS. 6A-6C, injection, massage, external compressions and partial retraction steps are repeated until the vein is completely treated.

Following the final injection and external compression, the apparatus is removed by first removing the second catheter 50. The collapsible occlusive balloon 30 is slowly deflated by removal of the closure device 42. Once the balloon 30 is fully deflated and collapsed, the first catheter 20 is removed followed by removal of the guide wire. The surgeon then closes and bandages the incision sites. The patient's leg remains in the elevated position ten to fifteen minutes. External encircling compressions are secured to the patient. Slow ambulation and discharge are complete after one to two hours. Compression bandages remain in place for twenty four hours, allowing the internal surface of the varicose vein to remain empty and completely adherent. External elastic bandages are applied daily for several subsequent days and ankle sutures, if used, are removed after four days.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An apparatus for segmental varicose sclerosis comprising:
a guide wire;
a flexible first catheter having a proximal end, a distal end, an outer surface, and a lumen area extending the length of the said first catheter;
a collapsible occlusive device and apparatus for deploying said device
a second catheter having a proximal end, a distal end, an outer surface, and a lumen area extending the length of said second catheter; and
a reservoir for dispensing a treating agent.

2. An apparatus for segmental varicose sclerosis comprising:
a guide wire;
a flexible first catheter positioned along the longitudinal axis of said guide wire whereby said first catheter has a proximal end, a distal end, an outer surface, and a lumen area extending the length of the said first catheter, said proximal portion fitted to receive an inflation device and a closure device;
a collapsible occlusive balloon attached to the distal end of said first catheter;
a second catheter second coaxially aligned with and longitudinally extending along the longitudinal axis of said first catheter, said second catheter having a proximal end, a distal end, an outer surface, and a lumen area extending the length of said second catheter, said lumen area larger than said first catheter, whereby said distal end of said second catheter is adapted for reciprocating longitudinal movement about said first catheter while maintaining sealing engagement with the periphery thereof; and
a reservoir for dispensing a treatment agent wherein said reservoir is fluidly coupled to said proximate end of said second catheter.

3. The apparatus for segmental varicose sclerosis of claim 2 wherein said guide wire is a Kirschner wire.

4. The apparatus for segmental varicose sclerosis of claim 2 wherein said lumen of said second catheter is fluidly coupled to radially and circumferentially spaced openings at said distal end of said second catheter.

5. The apparatus for segmental varicose sclerosis of claim 4 wherein said inflation device is a syringe, pump, bulb, valve, inlet ports or generator.

6. The apparatus for segmental varicose sclerosis of claim 5 wherein said closure device is an indwelling plug, ligature, or plastic adhesive.

7. The apparatus for segmental varicose sclerosis of claim 6 wherein said reservoir for dispensing a treatment agent is a syringe, pump, bulb, valve, inlet ports or generator.

8. The apparatus for segmental varicose sclerosis of claim 7 wherein said treatment agent is a sclerosing agent.

9. The apparatus for segmental varicose sclerosis of claim 8 wherein said first catheter receives a substance from said inflation device whereby receipt of said substance expands said collapsible occlusive balloon.

10. The apparatus for segmental varicose sclerosis of claim 9 wherein said substance is a gas.

11. The apparatus for segmental varicose sclerosis of claim 9 wherein said substance is a liquid.

12. The apparatus for segmental varicose sclerosis of claim 9 wherein said second catheter receives said treatment agent from said reservoir wherein said treatment agent is dispensed to a designated treatment area through said lumen which is fluidly coupled to said one or more radially and circumferentially spaced openings.

13. The apparatus for segmental varicose sclerosis of claim 2 further providing said first catheter having two lumens, said first lumen adapted to receive said guide wire and said second lumen adapted to receive a substance from said inflation device whereby receipt of said substance expands said collapsible balloon.

14. The apparatus for segmental varicose sclerosis of claim 2 further providing a vein sclerosing catheter assembly whereby said first catheter and said second catheter is pre-assembled.

15. A method of segmentally sclerosing varicose veins in a patient in need thereof comprising the steps of:
(a) forming a means of ingress into the skin and vein;
(b) percutaneously inserting a guide wire into said skin and vein;
(c) percutaneously inserting a first flexible catheter having a collapsible occlusive device into said vein;
(d) inflating and maintaining collapsible occlusive balloon;
(e) percutaneously inserting the distal end of a second catheter over said first catheter whereby said second catheter coaxially aligns with said first catheter and moveably extends over said first catheter.
(f) dispensing treatment agent;
(g) removing said apparatus; and
(h) closing said ingress site and applying post operative treatment whereby the length of the vein is sclerosed.

16. A method of segmentally sclerosing varicose veins in a patient in need thereof comprising the steps of:
(a) forming a means of ingress into the skin and saphenous vein;
(b) percutaneously inserting guide wire within the saphenous vein and passing said guide wire proximally within the saphenous vein to the femoral vein;
(c) percutaneously inserting a first flexible catheter along the longitudinal axis of said guide wire to the intersection of said patient's saphenous vein and femoral vein whereby said first catheter has a proximal end, a distal end, an outer surface, and a lumen area extending the length of the said first catheter, said proximal portion fitted to receive an inflation device and a closure device wherein said first catheter further having a collapsible occlusive balloon attached to the distal end;
(d) expanding said collapsible occlusive balloon whereby said first catheter receives an expanding substance from said inflation device, said substance being fluidly coupled to said balloon and causing said balloon to inflate thereby occluding blood flow of the vein;
(e) maintaining expansion of said collapsible occlusive balloon;
(f) percutaneously inserting a second coaxially aligned with and longitudinally extending along the longitudinal axis of said first catheter, said second catheter having a proximal end, a distal end, an outer surface, and a lumen area extending the length of said second catheter, said lumen area larger than said first catheter, whereby said distal end of said second catheter is adapted for reciprocating longitudinal movement about said first catheter while maintaining sealing engagement with the periphery thereof;
(g) dispensing treating agent from a reservoir fluidly coupled to said proximate end of said second catheter whereby said agent is dispensed through said lumen, said lumen fluidly coupled to radially and circumferentially spaced openings at said distal end of said second catheter;
(h) massaging the area;
(i) partially retracting said second catheter relative to said first catheter toward the proximate end;
(j) applying external compression dressings to the dispensing site;
(k) repeating steps (g), (h), (i) and (j) until the vein is treated;
(l) removing said apparatus in a step wise manner, the steps comprising (1) removal of second catheter, (2) causing said inflated occlusive balloon to deflate, (3) removal of said first catheter and (4) removal of said guide wire.
(m) closing said ingress site and applying post operative treatment whereby the length of the vein is sclerosed.

17. The method of segmentally sclerosing varicose veins in a patient in need thereof according to claim 16 wherein the guide wire is a Kirschner wire

18. The method of segmentally sclerosing varicose veins in a patient in need thereof according to claim 16 wherein said first catheter and said second catheter may be provided as a vein sclerosing assembly which is pre-assembled prior to percutaneous placement.

19. A kit for sclerosing a vein comprising:
a guide wire;
a first inflation catheter having one or more lumens;
a second treatment dispensing catheter;
a collapsible occlusive device and means for deploying and maintaining said device;
a treatment dispensing reservoir.

20. The kit of claim 19 wherein the guide wire is K-wire.

21. The kit of claim 20 wherein said collapsible occlusive device is an inflatable balloon.

22. The kit of claim 21 wherein said means for deploying said device is a syringe, pump, bulb, valve, or generator.

23. The kit of claim 22 wherein said means for maintaining said device is a indwelling plug, ligature, or plastic adhesive.

24. The kit of claim 23 wherein said treatment dispensing reservoir contains a sclerosing agent.
